(19) United States
(12) Patent Application Publication
(10) Pub. No.: US 2008/0139887 A1
(43) Pub. Date: Jun. 12, 2008

(54) Endoscopic working channel and method of making same

(75) Inventor: Craig Fitzpatrick, Phoenix, AZ (US)

Correspondence Address: LaValle D. Ptak, 28435 N. 42nd St., Ste. B, Cave Creek, AZ, 85331, US

(73) Assignee: International Polymer Engineering, Inc.

(21) Appl. No.: 11/636,270

(22) Filed: Dec. 7, 2006

(51) Int. Cl. A61B 1/00; B21D 35/00

(52) U.S. Cl. 600/153; 29/469.5

(57) ABSTRACT

An endoscopic working channel is made of an inner polytetrafluoroethylene tube having a spiral wrap of wire over it with an outer expanded polytetrafluoroethylene tube bonded over the wire to the inner polytetrafluoroethylene tube.

### RELATED PATENT

This patent application is related to the subject matter of U.S. Pat. No. 5,885,209, assigned to the same assignee as this application.

### BACKGROUND

The device of the present invention relates generally to the field of endoscopy, which includes the use of tubular structures inserted intraluminally into a mammalian body cavity for visualizing, biopsing, and treating tissue regions within the mammalian body. Most endoscopes currently include at least one of a plurality of working channels which extend along the length of the endoscope to provide access to body tissue within the mammalian body cavity. These working channels typically include a rigid non-bendable section and a flexible bendable section. The working channels allow for air insufflation, water flow, suction, and biopsies. Conventional endoscopes utilize a wide variety of materials for the working channels, but all conventional endoscopes require the endoscopic working channel to be an integral part of the endoscope.

Because endoscopes are subjected to repeated use and are required to follow tortuous pathways within the body, a frequent cause of failure of the endoscope working channel is the bending, kinking or fracture of a section of the working channel. This renders the endoscope useless until it is repaired. Unfortunately, repair of the endoscopic working channel requires disassembly of the endoscope and replacement of the endoscope working channel.

The endoscopic working channel of U.S. Pat. No. 5,885,209 is designed to be retrofitted as a replacement bendable section of the working channel of an endoscope. The structure of the endoscopic working channel of U.S. Pat. No. 5,885,209, however, is relatively complex and is relatively expensive to manufacture.

It is desirable to provide an improved endoscopic working channel.

### BRIEF DESCRIPTION OF THE DRAWINGS

FIG. 1 is a perspective view of an embodiment of the invention partially cut away to illustrate its structure;

FIG. 2 is a cross-sectional view of the device of FIG. 1 taken along a plane through the central axis of the device of FIG. 1; and

FIG. 3 is a process flow diagram illustrating a method used to manufacture the device of FIGS. 1 and 2;

### DETAILED DESCRIPTION

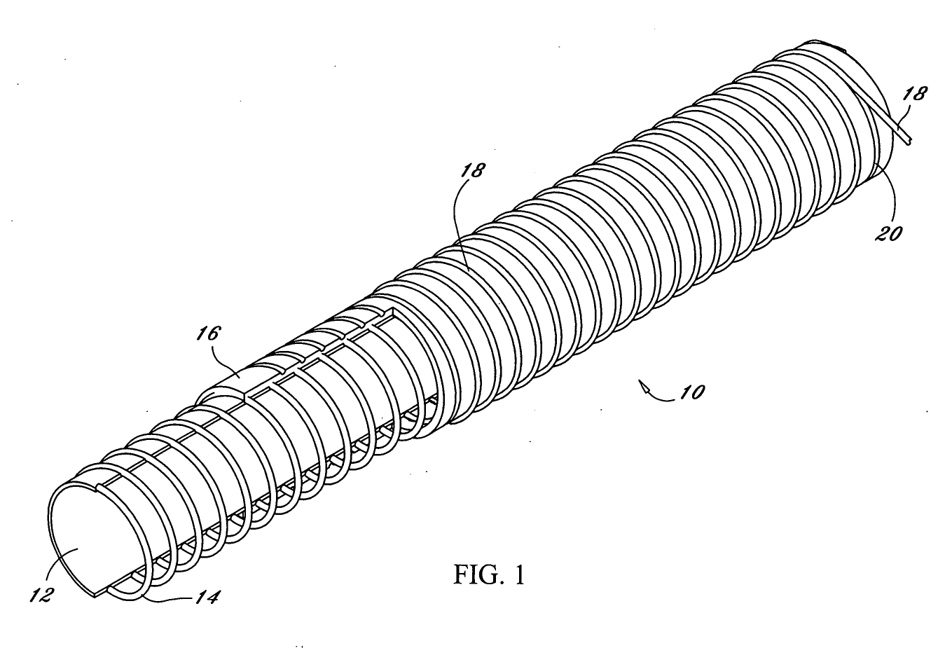

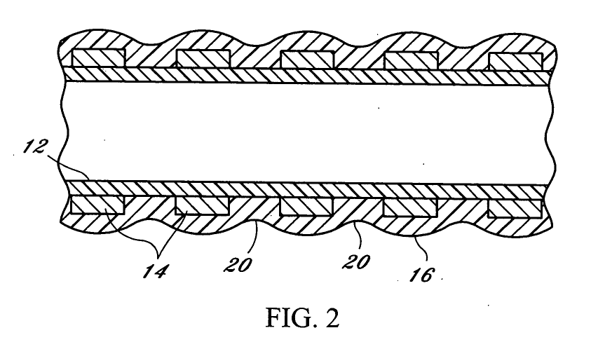

Reference now should be made to the drawings, in which the same reference numbers are used throughout the different figures to designate the same or similar components. FIGS. 1 and 2 are directed to an embodiment of an endoscopic working channel which includes an inner tubular structure or tube 12 fabricated from sintered, non-expanded or non-porous polytetrafluoroethylene (PTFE). Typically, the density of this inner tubular structure is in the range of 1.8 to 2.2 g/cc. A typical wall thickness of the inner PTFE structure is between 0.0045 inches and 0.007 inches for use as an endoscopic working channel. Directly over this inner PTFE layer 12 is a secondary layer in the form of a flat (rectangular cross section) wire 14 which is helically wrapped around the non-porous PTFE layer in a single spiral wrap. This wire typically is made of stainless steel. The wire 14 functions as a spring and also provides radial support to the completed tubing during flexion. The wire 14 also provides compression resistance as well as radial support during subsequent manipulation and internal pressurization of the tubing when it is placed in use.

A final or third layer of the structure shown in FIG. 1 is comprised of an outer (external) convoluted tube 16 made of expanded polytetrafluoroethylene (ePTFE) which is layered over the stainless steel wire 14 and the inner PTFE tube 12. The convolutions shown by the spiral depressions or valleys 20 (most clearly shown in FIG. 2) provide additional radial support during flexion, provide a bonding capability to the inner tube 12 which encapsulates the wire 14, and add a highly lubricious exterior surface.

The completed three-layer structure shown in FIGS. 1 and 2 has a highly lubricious inner and outer surface capable of a tight bend radius and a relatively low wall profile. The wire 14 provides added resistance to kinking. In addition, the completed structure is chemical resistant and is resistant to wear or collapse during repeated flexion.

Although the partially cut away view of FIG. 1 and the cross-sectional view of FIG. 2 may not illustrate it, the finished product has the opposing ends of the tubes 12 and 16 cut co-planar to a plane which is perpendicular to the common central axis of the inner and outer tubes 12 and 16. One or both of the opposing ends of the finished product may be chemically etched using an etcher suitable for use with polytetrafluoroethylene (PTFE), such as that sold under the trademarks “FLUROETCH” or “TETRAETCH” (W.L. Gore Associates).

Chemical etching facilitates subsequent adhesive bonding of the etched end with the tip of the endoscope. The end of the working channel which is intended to be the distal end of the channel may be chemically etched in order to increase the capacity of the tubes 12 and 16 to accept an adhesive bond for the distal section of an endoscope. The second or opposite end of the tubes 12 and 16, specifically that end which is intended to be the proximal end of the endoscope working channel, is not etched, as it typically is mechanically coupled to a proximal section of an endoscope. When the endoscope working channel depicted in FIGS. 1 and 2 is intended to be positioned in an intermediate region of the working channel, both the first and second ends of the endoscope working channel shown in FIGS. 1 and 2 preferably are chemically etched to increase their capacity to be adhesively bonded to the pre-existing working channel of the endoscope.

In order to form the convolutions, illustrated most clearly in FIG. 2 in the form of the spiral valleys 20 in the outer or ePTFE layer 16, a second spiral wire wrap is effected after the placement of the outer ePTFE tube 16 over the wire 14 and inner tube 12 has been completed. The spacing between adjacent turns of the outer wrap formed by the wire 18 shown in FIG. 1 is the same as the spacings between the adjacent turns of the inner or encapsulated spring wire 14, but offset; so that the winding of the wire 18 over the exterior surface of the ePTFE layer 16 is in the space between (although separated by the thickness of the tube 16) of adjacent turns of the inner wire 14. This causes the pressure depressions in the regions or valleys 20, illustrated in both FIGS. 1 and 2 to be effected.

After the outer wire 18 has been helically wound in place, the assembly, including the wire 18, is heated to a temperature above the sintering point of the inner and outer tubes 16. Once this has been done, the entire assembly is allowed to cool. The compression of the outer wire 18 in the regions 20 presses the inner diameter of the outer tube 16 into contact with the outer diameter of the inner tube 12, and into intimate contact with the surface of the inner helical wire 14. During sintering, a heat bonding of the tubes 12 and 16 takes place where they contact one another between adjacent turns of the wire 14. At the same time, the wire 14 is firmly encapsulated or sandwiched between the two tubes 12 and 16 as a result of the bonding which takes place. The typical melting point for the materials which are described is around 320° C. It also should be noted that while a sintering operation is effected as a final step in a sub-assembly of a working channel, the inner, non-porous PTFE tube 12 may be in the form of a sintered tube 12 at the inception of manufacture of the product, prior to the final sintering step which bonds the two tubes 12 and 16 to one another.

After the assembly illustrated in FIG. 1 has been allowed to cool, the wire 18 is removed, leaving the depressions 20 in the outer ePTFE tube 20 in place to form a convoluted exterior, as described previously. The wire 18 is discarded and does not form a part of the completed final assembly. The wire 14, however, remains encapsulated within the assembly, and substantially strengthens the finished tubular assembly to reduce kinking and to allow repeated flexion at relatively tight or small radii. It should be noted that the wrapping of the outer wire 18 during the manufacturing of the tube is placed on the exterior of the tube 16 with sufficient force to create a depression in the exterior of the tube 16 to a depth of approximately 0.001 inch. As mentioned above, the wire wrap, using the wire 18 during manufacturing, creates the depressions 20 in the exterior of the external tube 16, which assist in the flexion of the tubing. It also serves to maintain the tubing diameters during processing, and finally, applies force to the outer ePTFE tube 16 to ensure contact with the inner PTFE tube between the wire wraps of the wire 14. This also facilitates bonding of the layers during the secondary processing or sintering (heating) step in manufacture.

As mentioned above, a range of wall thickness which has been found satisfactory for the inner or non-porous PTFE tubing is between 0.0045 inches and 0.007 inches, with the thickness of the inner tubing 12 ranging from between 0.004 to 0.006 inches. For the wire 14, stainless steel wire has been found suitable, with a width of between 0.010 inches and 0.030 inches, and a thickness (radial dimension) of 0.001 inches to 0.004 inches. The wire pitch or spacing may be as low as the width of the exterior wire diameter of the wire 18, which is on the order of 0.006 inches; and the maximum spacing between adjacent turns of the encapsulated stainless steel wire 14 may be as high as 10:1. The wire spacing between adjacent turns of the spirally wound inner stainless steel wire 14 is dependent upon the characteristics of the other components of the three-layer structure. These characteristics include the inner PTFE tubing thickness of the tube 12, the total wall thickness, the wire diameter and width, and the density of the outer ePTFE tubing 16, along with its wall thickness, in order to meet the desired flexural/cycle life intended for the use to which the finished product is to be placed.

The outer ePTFE tube 16 obviously has an internal diameter which is in a close relationship to the external diameter of the inner tube 12. The wall thickness of the tubing is dependent on the wall thickness of the inner PTFE tube 12. A range of wall thicknesses of the two layers is for the ePTFE layer 16 to be a 2:1 ratio between the inner PTFE tube wall thickness to the outer ePTFE tube 16. A maximum outer tube 16 to inner tube 12 wall thickness is 8:1 ratio. For example, if the inner PTFE tube 12 has a thickness of 0.004 inches, then the wall thickness of the outer ePTFE tube 16 ranges between 0.008 inches and 0.0032 inches. These are not critical dimensions, since the thickness of the exterior tube 16 may be varied, but obviously must be within the concept of the design of its intended use or implementation to maintain a low profile tubing with high flexibility without kinking. The ranges given are practical ranges for most applications.

The outer ePTFE tube 16 is made up of a matrix of nodes and fibers which run the length of the tubing. The nodes are oriented perpendicular to the fiber, which run longitudinally down the tubing. The nodes are relatively a static or solid portion of the ePTFE micro-structure, while the fibers which interconnect the nodes are collapsible, allowing the tubing to undergo longitudinal compression and elongation without dimensional changes, much like the performance of a spring. In fact, the inner stainless steel wire 14 functions as a supplement to this spring-like action.

It is the ratio of the length of the fibers and the width of the nodes that allow various amounts of flexion in ePTFE tubing. Longer fiber lengths and smaller nodes provide tubing with high flexibility and low radial support. Since the length of the fibers relates to the amount of open space in the micro-structure of ePTFE tubing, the relationship can be expressed in tubing volume. The relationship between fiber lengths is inverse to the density of the tubing. For example, a tube 16 made of ePTFE with a 25 micron fiber length could have a volume density of 0.06 g/cc, while a tube with a 10 micron fiber length would have a density of 1.2 g/cc or higher. The volume density range for ePTFE to function in the design of the product described and shown in FIGS. 1 and 2, can range from 0.2 to 1.9 g/cc.

The overall thickness of the finished structure, which is shown in cross section in FIG. 2, typically is between 0.014 inches and 0.058 inches. The finished product has a highly lubricious interior layer which is determined by the coefficient of friction of the PTFE material in the tube 12. Chemical resistance of PTFE to most acids, bases, alcohols and so forth exists; and the temperature resistance of PTFE up to 300° Centigrade (below the sintering or melting point) occurs. As mentioned above, the outer wall of the inner PTFE tube 12 and the inner wall of the outer ePTFE tube 16 are bonded together via temperature and pressure, and require no adhesives or chemicals to create the bond between the two tubular layers.

It has been found that completed units have an average cycle life of 5,000 cycles along the minimum bend radius of the completed tubing. In addition, completed units have been found to be capable of up to 80 PSI interior air pressures without more than 5% radial diameter deflection or leaking. Once again, the encapsulated inner wire member 14 improves this stability over structures which do not include the reinforcement of the wire 14.

Within the range of the structures which have been described, it also has been found that there are no more than three percent radial deflection at a one-half inch radius. This has been found to approximate a maximum bend condition which may occur during use of the device.

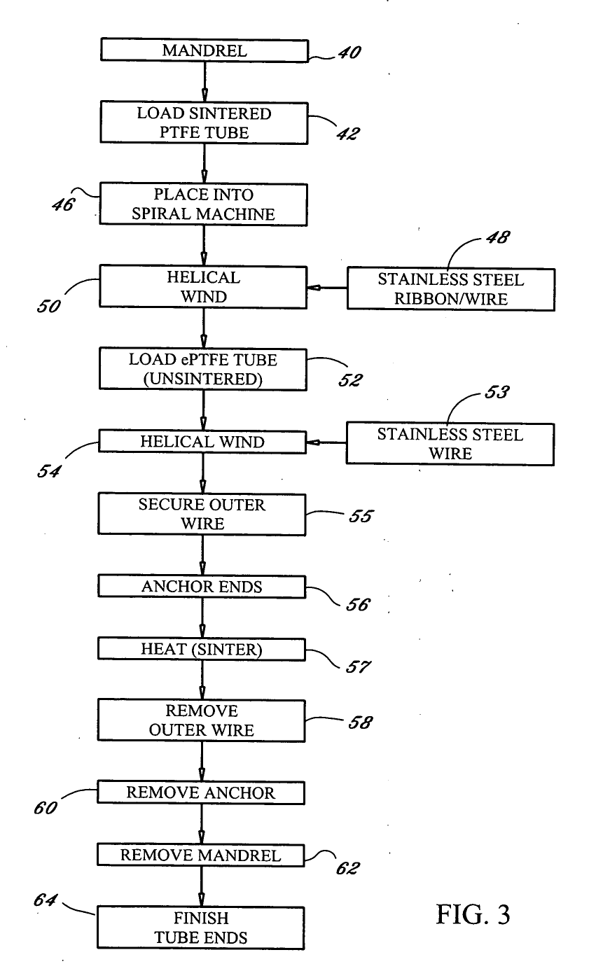

Reference now should be made to FIGS. 1 and 3 in explaining the manner in which the endoscope working channel of the embodiment of FIGS. 1 and 2 is manufactured. The first step 40 in FIG. 3 is to provide a mandrel (not shown) which is either a rod or a tube made of stainless steel, brass or aluminum, having a length which preferably is greater than the length of the finished endoscope working channel to be made by the process. The mandrel is a straight rod, the ends of which extend beyond the length of the endoscope working channel. The rod is designed to be mounted for rotation in a conventional spiral winding machine.

Before placing the assembly in a winding machine, however, the inner PTFE tube 12 (sintered or un-sintered) is loaded onto the rod (step 42 of FIG. 3) simply by sliding it onto the mandrel from one end toward the other. Once the inner PTFE tube 12 is in place on the mandrel, the mandrel with the tube 12 on it is placed into a spiral machine as shown at step 46 in FIG. 3. After placement of the mandrel or rod with the tube 12 on it into the spiral machine at step 46, windings at step 50 (FIG. 3) of the stainless steel wire 14 from a source 48 are tightly wound in a spiral or helical pattern on the outer diameter of the tube 12, from one end of the assembly (for example, the left-hand end as shown in FIG. 1) toward the right-hand end onto the mandrel. The winding of the wire 14 is done with sufficient pressure to firmly grip the exterior diameter of the tube 12. After the wire 14 has been wound on the tube 12, the outer, un-sintered ePTFE tube 16 is loaded onto the mandrel at step 52 of FIG. 3 by simply sliding it onto the mandrel 10 from one end toward the other, over the tube 12 and the spirally wound wire 14. It should be noted that the inner diameter of the tube 16 is equal to or slightly greater than the outer diameter of the tube 12 covered by the spiral winding of the wire 14.

After the tube 16 has been loaded as described above, the assembly once again is operated in the spiral machine, as shown at step 54, to wind the outer wire 18 from a supply 53 in the helical or spiral winding at step 54 to place the outer winding over the spaces between adjacent turns of the encapsulated wire 14. The wire 18 from the supply 53, while indicated as stainless steel wire, may be brass, aluminum or ribbon wire of any desired type. It is tightly wound in a spiral or helical pattern over the exterior of the tube 16, over one end of the assembly (again, the left-hand as shown in FIG. 1) toward the right-hand end onto the end of the mandrel (not shown). The wire 18 which is wound at step 54 of FIG. 3 may have either a circular cross section or a rectangular cross section. The winding of the wire or ribbon wire 18 is done with sufficient pressure so as to compress the portions of the ePTFE tube 16 beneath the wire 18 as the winding takes place with non-compressed spaces between adjacent turns of the wire 18. This in turn supplies pressure between the inner diameter of the tube 16 and the outer diameter of the inner PTFE tube 12 (in the spaces between adjacent turns of the wire 14). This also produces some compression of the pores of the outer ePTFE tube 16 beneath each of the turns of the wire wrap 18.

The wire wrap 18 is shown in FIG. 1, and serves both to maintain the dimensional aspect of the tubing 16 and provide some radial compression to assist in the bonding of the two tubes 12 and 16 together. In addition, the wire 18 is made of heat conductive material (particularly when stainless steel is used); so that the wire 18 facilitates the subsequent heating steps. Other wire shapes in addition to those described here also could be used.

After the spiral winding of the wire 18 at step 54, the wire is secured at the ends of the mandrel at 55 (FIG. 3) by means of a removable tape or any suitable material (not shown), to hold it in place during the final sintering steps of the method of fabrication of the endoscope working channel. This securing or anchoring is shown at step 56 in FIG. 3. Also, in addition to the spiral wire or helical wire 18, brass wire or rings (not shown) may be secured around the exterior of the assembly over the outer diameter of the tube 16 adjacent both ends to further secure the wire wrap 18 to the tubing 16, and to prevent longitudinal retraction of the outer tubing 16 during the next processing step.

As mentioned above, the sintering point of polytetrafluoroethylene (PTFE) and expanded polytetrafluoroethylene (ePTFE) is approximately 320° Centigrade. Once the ends of the wire 18 and the of the tubing 16 are secured at step 56 (FIG. 3), the entire assembly, including the mandrel on which it is being formed, is removed from the winding machine and placed in a heat sintering oven (step 57 of FIG. 3), which may be in the form of a convection air oven or a furnace at a processing temperature sufficiently high to exceed the 320° Centigrade sintering point of the expanded polytetrafluoroethylene (ePTFE) material. Induction heating ovens also may be used providing the temperatures to which the expanded polytetrafluoroethylene (ePTFE) is subjected exceed the 320° Centigrade sintering temperature. The time duration for this sintering process of the tube 16 (or of both tubes 12 and 16) is approximately one to two minutes duration per foot of the assembly. This time, however, may be varied in accordance with the particular parameters of the oven used and the manner in which heat is applied to the assembly during the sintering process.

After the sintering process has been completed at step 57, and the assembly shown in FIG. 1 has been allowed to cool, the anchors are removed (at step 60) from both ends of the assembly. The wire 18 is unraveled and discarded, as shown at step 58 in FIG. 3. The completed assembly then has the configuration shown in FIGS. 1 and 2. At this time, the mandrel is removed at step 62; and the two ends of the assembly are finished at step 64 in the manner described previously.

Although the embodiment which has been described above includes the winding of a wire 18 and its subsequent removal to form a convoluted exterior configuration of the completed assembly, some applications may require an exterior surface which is relatively smooth, that is not convoluted. In such a case, no wire 18 would be wound around the exterior of the expanded polytetrafluoroethylene tube 16 prior to the final sintering process. If such convolutions are not desired, steps 53,54,55, and 58 of the process described above and illustrated in FIG. 3 would be eliminated. The remaining steps, however, still apply to form a completed assembly having a configuration similar to that of FIG. 2, but without any of the convolutions or valleys 20 shown in FIG. 2.

The foregoing description of an embodiment of the invention is to be considered as illustrative and not as limiting. Various changes and modifications will occur to those skilled in the art for performing substantially the same function, in substantially the same way, to achieve substantially the same result without departing from the true scope of the invention as defined in the appended claims.

What is claimed is:

1. An endoscopic working channel capable of retrofit into a pre-existing endoscope including in combination: an inner sintered polytetrafluoroethylene tubular member having an internal diameter and an external diameter, and having first and second opposing ends; a spiral wrap of metal wire over the external diameter of the inner tubular member; an outer tubular member over the spiral wire wrap and the inner tubular member and made of sintered expanded polytetrafluoroethylene with first and second opposing ends, and having an internal diameter selected to cause an intimate contact with the spiral wire wrap and the inner tubular member, with the outer expanded polytetrafluoroethylene tubular member bonded to the inner polytetrafluoroethylene tubular member between adjacent turns of the spiral wire wrap.

2. The endoscopic working channel according to claim 1 wherein the outer tubular member is heat bonded to the inner tubular member.

3. An endoscopic working channel according to claim 2 wherein the external expanded polytetrafluoroethylene tubular member is sintered onto a previously sintered polytetrafluoroethylene inner tubular member.

4. An endoscopic working channel according to claim 3 wherein the outer expanded polytetrafluoroethylene tubular member has a convoluted outer surface.

5. An endoscopic working channel according to claim 4 wherein the convolutions in the outer surface of the outer expanded polytetrafluoroethylene tubular member are in a continuous spiral or helical pattern of raised portions separated by valleys, the raised portions of which lie over the spiral wire wrap and the valleys of which are located between adjacent turns of the spiral wire wrap.

6. An endoscopic working channel according to claim 5 wherein the spiral wire wrap is stainless steel.

7. An endoscopic working channel according to claim 6 wherein the spiral wire wrap is configured with a rectangular cross section.

8. An endoscopic working channel according to claim 1 wherein the spiral wire wrap is stainless steel.

9. An endoscopic working channel according to claim 8 wherein the spiral wire wrap is configured with a rectangular cross section.

10. An endoscopic working channel according to claim 1 wherein the outer expanded polytetrafluoroethylene tubular member has a convoluted outer surface.

11. An endoscopic working channel according to claim 10 wherein the convolutions in the outer surface of the outer expanded polytetrafluoroethylene tubular member are in a continuous spiral or helical pattern of raised portions separated by valleys, the raised portions of which lie over the spiral wire wrap and the valleys of which are located between adjacent turns of the spiral wire wrap.

12. An endoscopic working channel according to claim 1 wherein the external expanded polytetrafluoroethylene tubular member is sintered onto a previously sintered polytetrafluoroethylene inner tubular member.

13. An endoscopic working channel according to claim 12 wherein the outer expanded polytetrafluoroethylene tubular member has a convoluted outer surface.

14. An endoscopic working channel according to claim 13 wherein the spiral wire wrap is stainless steel.

15. An endoscopic working channel according to claim 1 wherein the spiral wire wrap is configured with a rectangular cross section.

16. A method of making an endoscopic working channel including: placing a first fixed length of tubing made of non-expanded polytetrafluoroethylene (PTFE) on a mandrel; spiral winding a wire about the exterior of the first fixed length of tubing; placing a second fixed length of tubing made of expanded polytetrafluoroethylene (ePTFE) over the first fixed length of tubing and the spiral winding of wire; spiral winding a second wire about the exterior of the second fixed length of tubing at the same pitch of the winding of the first spiral wire and located over the spacings between adjacent turns of the first spiral wire; heating the assembly to bond the first and second fixed lengths of tubing together between adjacent turns of the first spiral wire; and removing the second spiral wire from the exterior of the second fixed length of tubing.

17. A method according to claim 16 further including removing the mandrel from the interior of the first fixed length of tubing after the second wire is removed from the exterior of the second fixed length of tubing.

18. The method according to claim 17 wherein spiral winding the first wire about the exterior of the first length of tubing comprises spiral winding the wire with a predetermined spacing between each turn of the spiral; and spiral winding the second wire about the exterior of the second fixed length of tubing comprises winding a second wire with the same predetermined spacing as the first wire between each turn of the spiral of the second wire.

19. The method according to claim 18 wherein winding the second wire about the exterior of the second fixed length of tubing comprises winding the second wire with sufficient pressure to compress the portions of the second fixed length of tubing located beneath the wire into constant contact with the first length of tubing between adjacent turns of the first wire.

20. The method according to claim 19 further including finishing the ends of the bonded first and second fixed lengths of tubing after removal of the mandrel.

21. The method according to claim 16 wherein winding the second wire about the exterior of the second fixed length of tubing comprises winding the second wire with sufficient pressure to compress the portions of the second fixed length of tubing located beneath the wire into constant contact with the first length of tubing between adjacent turns of the first wire.

22. The method according to claim 16 wherein spiral winding the first wire about the exterior of the first length of tubing comprises spiral winding the wire with a predetermined spacing between each turn of the spiral; and spiral winding the second wire about the exterior of the second fixed length of tubing comprises winding a second wire with the same predetermined spacing as the first wire between each turn of the spiral of the second wire.

23. The method according to claim 16 further including finishing the ends of the bonded first and second fixed lengths of tubing after removal of the mandrel.

24. A method of making an endoscopic working channel including: placing a first length of tubing made of sintered non-expanded polytetrafluoroethylene (PTFE) on a mandrel; spiral winding a metal wire with predetermined spacings between adjacent turns of the wind on the first fixed length of tubing; placing a second fixed length of tubing made of un-sintered expanded polytetrafluoroethylene (ePTFE) over the first fixed length of tubing and the spiral wound wire to form an intermediate assembly; and heating the intermediate assembly to bond the first and second fixed lengths of tubing together between the adjacent turns of the spiral wound wire.

25. The method according to claim 24 wherein placing the first fixed length of tubing on the mandrel comprises sliding the first fixed length of tubing over a mandrel having a length greater than the length of the first fixed length of tubing.

26. A method according to claim 25 further including removing the mandrel from the interior of the first fixed length of tubing after the first and second fixed lengths of tubing are bonded together.

27. The method according to claim 26 further including finishing the ends of the bonded first and second fixed lengths of tubing after removal of the mandrel.

28. A method according to claim 24 further including removing the mandrel from the interior of the first fixed length of tubing after the first and second fixed lengths of tubing are bonded together.

29. The method according to claim 24 further including finishing the ends of the bonded first and second fixed lengths of tubing after removal of the mandrel.
